(19) United States
(12) Patent Application Publication
(10) Pub. No.: US 2011/0236358 A1
(43) Pub. Date: Sep. 29, 2011

(54) COMPOSITION COMPRISING ISOFLAVONES

(76) Inventor: Stefano Sala, Trento (IT)

(21) Appl. No.: 12/998,846

(22) PCT Filed: Dec. 10, 2009
(86) PCT No.: PCT/EP2009/066867

§ 371 Date: Jun. 8, 2011

(30) Foreign Application Priority Data
Dec. 12, 2008 (IT) PD2008A000367

(51) Int. Cl. A61K 35/74; A61K 31/7034; A61K 36/258; A61K 36/708; A61P 5/24; A61P 25/24; A61P 25/22; A61P 25/32; A61P 3/04

(52) U.S. Cl. 424/93.44; 514/27; 424/93.45; 424/93.4; 424/728

(57) ABSTRACT

A composition comprising at least one isoflavone, phloridzin and one or more probiotics. A composition as described herein is also provided for internal use as a natural alternative to hormone replacement therapy and for treating disorders such as menopause and correlated symptoms, alcoholism, stress, insomnia, obesity and cellulite.

The present invention refers to a composition comprising phytoestrogen isoflavones characterized by improved absorption and bioavailability of the isoflavones.

### BACKGROUND OF THE INVENTION

Several products characterized by the presence of isoflavones as active ingredients are currently commercially available. Isoflavones are a subclass of polyphenolic cyclic compounds that belong to the wider class of flavonoids. Isoflavones can be obtained from natural sources and by chemical synthesis. With respect to most flavonoids, isoflavones are distinct in that they are colorless and have a rather limited taxonomic distribution, since they are present exclusively in Leguminosae and in Iridaceae.

In plants, isoflavones have several biochemical roles, acting most of all as phytoestrogens but also as antioxidants and antibacterials. On a medical level, some isoflavones, such as for example genistein, daidzein and coumestrol, have demonstrated to be weak estrogen agents. Moreover, genistein, the major isoflavone in soybean, has demonstrated chemopreventive properties against cancer, although at present the mechanism of action on which this effect is based has not yet been clarified.

The taking of isoflavones by an individual is regulated by law, which limits the daily quantity to 80 mg. This limit is a protective measure which can, however, leads to low effectiveness or total ineffectiveness of a product whose active ingredient is an isoflavone. Since the actual bioavailability of isoflavones in fact depends on many variables, it can change even very significantly from one individual to another. Therefore, the need to limit the taking of isoflavones to 80 mg/day can be a substantial disadvantage where the actual bioavailability of the isoflavones provided by a product is not sufficient to reach the levels of isoflavones that are needed for these substances to be able to have a beneficial effect.

There is, therefore, the need to develop new products based on isoflavones whose composition is studied in order to overcome the problems described above, improving the bioavailability of isoflavones for an equal dosage.

### SUMMARY OF THE INVENTION

The aim of the present invention is to provide a new composition based on isoflavones whose formulation allows high absorption and better bioavailability of isoflavones.

Within this aim, an object of the invention is to provide a composition that can provide a natural alternative to hormone replacement therapy.

Another object of the invention is to provide a composition based on isoflavones that can be used to treat a disorder selected from the group constituted by menopause and its symptoms, alcoholism, stress, insomnia, obesity and cellulite.

Another object of the invention is to provide a composition based on isoflavones that is highly reliable, relatively easy to provide and at competitive costs.

This aim, as well as these and other objects that will become better apparent hereinafter, are achieved by a composition comprising at least one isoflavone, phloridzin and one or more probiotics.

The aim and objects of the invention are also achieved by a composition as described herein for internal use as a natural alternative to hormone replacement therapy.

Moreover, the aim and objects of the invention are also achieved by a composition as described here for the treatment of a disorder selected from the group constituted by menopause and correlated symptoms, alcoholism, stress, insomnia, obesity and cellulite.

Further objects, characteristics and advantages of the invention will become better apparent from the following detailed description.

### DESCRIPTION OF THE PREFERRED EMBODIMENTS

In one of its embodiments, the present invention relates to a composition which comprises isoflavones in combination with other active ingredients, which thanks to their synergy are capable of optimizing the assimilation and bioavailability of the isoflavones when the composition is taken by an individual. In particular, the additional ingredients of the present composition that are added to the isoflavones are phloridzin and one or more probiotics.

Preferably, in the preparation of the composition according to the present invention the at least one isoflavone that is used as raw material can have a titer from 1% to 100% by weight; the phloridzin can have a titer from 1% to 100% by weight; and the one or more probiotics can have a titer from 50×10⁹ to 200×10⁹ ferments/g.

In one embodiment of the composition according to the invention, the at least one isoflavone can be in a quantity from 0.001% to 99.998% by weight, the phloridzin can be in a quantity from 0.001% to 99.998% by weight, and the one or more probiotics can be in a quantity from 0.001% to 99.998% by weight. In a preferred embodiment, the at least one isoflavone can be in a quantity from 0.15% to 0.90% by weight. In another preferred embodiment, the phloridzin can be in a quantity from 0.005% to 0.090% by weight. In a further preferred embodiment, the one or more probiotics can be in a quantity from 0.004% to 0.11% by weight.

As mentioned earlier, isoflavones are vegetable-derived substances which in plants perform various biochemical functions, including those of antioxidants, phytoestrogens or antibacterials. The composition according to the present invention comprises at least one isoflavone, preferably several isoflavones which may even be mutually different. Preferably but not exclusively, the isoflavones are selected from the group constituted by puerarin, daidzin, daidzein, genistin, genistein and mixtures thereof.

In one embodiment, the at least one isoflavone that is part of the composition described here can be extracted from a plant selected from the group constituted by Pueraria lobata (kudzu), Glycine max (soybean) and Trifolium pratense (red clover).

The second component of the composition according to the invention is constituted by phloridzin (or phlorizin), a natural glycoside which is present in several fruit-bearing trees, such as for example the apple tree, and can be extracted from sources such as the bark, the roots and the peel of the fruit of plants. In the composition according to the present invention, phloridzin increases the absorption of isoflavones, thus allowing an individual to whom the composition is administered to assimilate the highest possible quantity of isoflavones.

The third component of the composition according to the present invention is represented by one or more probiotics. Probiotics are dietary supplements which contain microorganisms, such as for example bacteria or yeasts, whose administration in adequate quantities provides beneficial effects to the subject who receives the administration. In the composition according to the invention, the probiotics act at the intestinal level, facilitating the bioavailability of the isoflavones.

In one embodiment of the present invention, the one or more probiotics are preferably but not exclusively selected from the group constituted by Lactobacillus acidophilus, Lactobacillus casei, Lactobacillus helveticus, Streptococcus thermophilus, Streptococcus lactis, Lactobacillus bulgaricus, Bifidobacterium bifidum, Bifidobacterium breve, Bifidobacterium infantis, Bifidobacterium longum, Lactobacillus plantarum, Lactobacillus rhamnosus, Lactobacillus salivarius, Lactobacillus lactis and mixtures thereof.

Preferably, the one or more probiotics of the composition according to the present invention can be either live or tyndallized.

In another embodiment of the present invention, the composition described here can further comprise other substances that have a beneficial action. These substances can be selected from the group constituted by minerals, vitamins, phytoextracts and prebiotics.

Preferably but not exclusively, the prebiotics can be selected from the group constituted by inulin, fructo-oligosaccharides (FOS), galacto-oligosaccharides (GOS), and mixtures thereof.

Preferably but not exclusively, the phytoextracts can be phytoextracts of plants selected from the group constituted by Crataegus monogyna, Melissa officinalis, Passiflora sp., Humulus lupulus, Hypericum perforatum, Panax ginseng, Salvia miltiorrhiza, Cimicifuga racemosa, Cassia angustifolia, Cassia nomame, Coffea arabica, Griffonia simplicifolia, Equisetum arvense, Taraxacum dens leonis, Sambucus nigra, Rheum officinalis, Rhamnus purshiana, Carica papaya and mixtures thereof.

The composition according to the present invention can take various forms in which it is administered to a subject. For example, the composition can be in liquid or solid form.

The solid administration forms can include capsules, tablets, pills, powders or granules. In these solid forms, the composition according to the invention may further comprise one or more suitable excipients normally used in the pharmaceutical and food sectors.

The liquid administration forms can instead include emulsions, solutions, suspensions, syrups or elixirs. In these liquid forms, the composition according to the invention can further comprise one or more suitable excipients, such as for example diluents, humectants, emulsifiers or suspension agents, which are normally used in the pharmaceutical and food sector.

In another aspect, the present invention relates to the use of the composition described here as a pharmaceutical and nonpharmaceutical product intended for internal use, for example as a natural alternative to hormone replacement therapy.

Generally, the expression “hormone replacement therapy” refers to a pharmacological treatment based on hormones that are normally produced by the female body, particularly by the ovaries. These hormones are mostly estrogens and are used both on their own and in association with progestinic hormones. For example, in women who have undergone hysterectomy and whose uterus has been removed surgically, hormone replacement therapy is based only on estrogens. The hormones used mainly for hormone replacement therapy are estradiol, estrone, estriol and ethynyl estradiol. During menopause, the production of these hormones decreases and characteristic symptoms, such as hot flushes, insomnia and vaginal disorders, can appear. Hormone replacement therapy seeks to correct this lack of hormones and consequently reduce the symptoms of menopause.

The present invention therefore relates also to a composition as described here for use as a natural alternative to hormone replacement therapy, utilizing therefore plant-derived products, such as isoflavones and phloridzin, as a replacement of estrogens.

Moreover, the present invention also relates to a composition as described here for the treatment of a disorder selected from the group constituted by menopause and correlated symptoms, alcoholism, stress, insomnia, obesity and cellulite.

In a preferred embodiment, the composition according to the invention for treating the menopause and its symptoms can be characterized in that it comprises, in addition to the three main active ingredients, minerals, vitamins and one or more phytoextracts of plants selected from the group constituted by Crataegus monogyna, Melissa officinalis, Passiflora sp., Humulus lupulus and Cimicifuga racemosa.

In another preferred embodiment, the composition according to the invention for treating alcoholism can be characterized in that it comprises, in addition to the three main active ingredients, one or more phytoextracts of plants selected from the group constituted by Humulus lupulus, Hypericum perforatum, Panax ginseng and Salvia miltiorrhiza.

In a further preferred embodiment, the composition according to the invention for treating obesity can be characterized in that it comprises, in addition to the three main active ingredients, one or more phytoextracts with draining and detoxifying properties which are useful in maintaining tissue tone and elasticity, one or more minerals, such as for example copper, selenium and iron, and one or more additional active ingredients, such as for example theanine and collagen. In particular, the draining and detoxifying phytoextracts can be one or more phytoextracts of plants selected from the group constituted by Cassia angustifolia, Cassia nomame, Coffea arabica, Griffonia simplicifolia, Equisetum arvense, Taraxacum dens leonis, Sambucus nigra, Rheum officinalis, Rhamnus purshiana and Carica papaya.

Moreover, in another aspect the present invention also relates to a composition as described here that can be used to facilitate the redistribution of the fatty mass or to improve breast tone.

In a preferred embodiment, the composition according to the invention for facilitating redistribution of the fatty mass can be characterized in that it comprises, in addition to the three main active ingredients, one or more of the draining and detoxifying phytoextracts, of the minerals and of the additional active ingredients used in the composition for the treatment of obesity described here.

Advantageously, the composition according to the present invention can therefore receive the addition, in all the formulations adapted to facilitate improvement of female problems related to hormone imbalance, so as to promote the slimming of women (in particular over 30 years of age), the improvement of breast tone and the improvement of skin blemishes, in particular cellulite.

Non-limiting examples of the composition according to the invention can be:

a composition for the treatment of insomnia, comprising isoflavones of Pueraria lobata (kudzu), phloridzin, Lactobacillus acidophilus, Lactobacillus casei, Lactobacillus thermophilus, phytoextract of Melissa Officinalis and phytoextract of Passiflora;

a composition for stress treatment, comprising isoflavones from Glycine max, phloridzin, Lactobacillus acidophilus, Lactobacillus casei, Lactobacillus thermophilus, phytoextract of Hypericum perforatum and phytoextract of Panax ginseng;

a composition for treating menopausal symptoms, comprising isoflavones from Pueraria lobata (kudzu), phloridzin, Lactobacillus acidophilus, Lactobacillus casei, Lactobacillus thermophilus and phytoextract of Crataegus monogyna;

a composition for treating alcoholism, comprising isoflavones from Trifolium pratense (red clover), phloridzin, Lactobacillus acidophilus, Lactobacillus casei, Lactobacillus thermophilus, phytoextract of Salvia miltiorrhiza and phytoextract of Humulus lupulus;

a composition for treating obesity or facilitating redistribution Of fatty mass, comprising isoflavones from Pueraria lobata (kudzu), phloridzin, Lactobacillus acidophilus, Lactobacillus casei and phytoextracts of Cassia angustifolia, Coffea arabica, Sambucus nigra and Griffonia simplicifolia.

In practice it has been found that the composition according to the invention fully achieves the intended aim, since the synergistic combination of isoflavones, phloridzin and probiotics allows to increase the absorption of isoflavones and leads to better bioavailability of this active ingredient.

Moreover, it has been found that the composition according to the invention, thanks to the higher absorption and the better bioavailability of isoflavones, can be used as hormone replacement therapy.

It has also been found that the composition according to the invention is suitable for preparing formulations which are optionally combined with various phytoextracts and is useful as a natural alternative to hormone replacement therapy and for the treatment of menopause and its symptoms, alcoholism, stress, insomnia, obesity and cellulite.

The composition thus conceived is susceptible of numerous modifications and variations, all of which are within the scope of the appended claims; all the details may further be replaced with other technically equivalent elements.

The disclosures in Italian Patent Application No. PD2008A000367 from which this application claims priority are incorporated herein by reference.

1-11. (canceled)

12. A composition comprising at least one isoflavone, phloridzin and one or more probiotics.

13. The composition according to claim 12, wherein the at least one isoflavone is in a quantity from 0.001% to 99.998% by weight, the phloridzin is in a quantity from 0.001% to 99.998% by weight, and the one or more probiotics are in a quantity from 0.001% to 99.998% by weight.

14. The composition according to claim 12, wherein the at least one isoflavone is selected from the group constituted by puerarin, daidzin, daidzein, genistin, genistein and mixtures thereof.

15. The composition according to claim 12, wherein the at least one isoflavone is extracted from a plant selected from the group constituted by Pueraria lobata (Kudzu), Glycine max (Soybean) and Trifolium pratense (Red Clover).

16. The composition according to claim 12, wherein the one or more probiotics are selected from the group constituted by Lactobacillus acidophilus, Lactobacillus casei, Lactobacillus helveticus, Streptococcus thermophilus, Streptococcus lactis, Lactobacillus bulgaricus, Bifidobacterium bifidum, Bifidobacterium breve, Bifidobacterium infantis, Bifidobacterium longum, Lactobacillus plantarum, Lactobacillus rhamnosus, Lactobacillus salivarius, Lactobacillus lactis and mixtures thereof.

17. The composition according to claim 12, further comprising one or more substances selected from the group constituted by minerals, vitamins, phytoextracts and prebiotics.

18. The composition according to claim 17, wherein the prebiotics are selected from the group constituted by inulin, fructo-oligosaccharides (FOS), galacto-oligosaccharides (GOS), and mixtures thereof.

19. The composition according to claim 17, wherein the phytoextracts are phytoextracts of plants selected from the group constituted by Crataegus monogyna, Melissa officinalis, Passiflora sp., Humulus lupulus, Hypericum perforatum, Panax ginseng, Salvia miltiorrhiza, Cimicifuga racemosa, Cassia angustifolia, Cassia nomame, Coffea arabica, Griffonia simplicifolia, Equisetum arvense, Taraxacum dens leonis, Sambucus nigra, Rheum officinalis, Rhamnus purshiana, Carica papaya and mixtures thereof.

20. The composition according to claim 12, wherein it is in liquid or solid form.

21. The composition according to claim 12 for use as a natural alternative to hormone replacement therapy.

22. The composition according to claim 12 for treating a disorder selected from the group constituted by menopause and correlated symptoms, alcoholism, stress, insomnia, obesity and cellulite.
